(19) United States
(12) Patent Application Publication
(10) Pub. No.: US 2026/0306743 A1
(43) Pub. Date: Oct. 8, 2026

(54) DRUG DELIVERY DEVICE

(71) Applicant: SANOFI, Paris (FR)

(72) Inventors: Nima Aghajari, Frankfurt am Main (DE); Uwe Dasbach, Frankfurt am Main (DE); Tim Glässer, Rüsselsheim (DE); Moritz Keim, Frankfurt am Main (DE); Matthias Rau, Rüsselsheim (DE); Michael Schabbach, Frankfurt am Main (DE)

(21) Appl. No.: 19/490,353

(22) PCT Filed: Jun. 5, 2024
(86) PCT No.: PCT/EP2024/065442

§ 371 Date: Dec. 4, 2025

(30) Foreign Application Priority Data
Jun. 5, 2023 (EP) 23315233.9

(51) Int. Cl. A61M 5/20; A61M 5/32; A61M 5/46

(52) U.S. Cl. CPC A61M 5/20; A61M 5/3295; A61M 5/46; A61M 2005/2013; A61M 2005/3267

(57) ABSTRACT

A drug delivery device is provided. The drug delivery device comprises: a housing for receiving a pouch within the housing, the pouch having collapsible walls enclosing a chamber, a dispensing side, and a remote side facing away from the dispensing side, and accommodating a drug within the chamber; a needle arrangement comprising at least one needle being configured to pierce the skin of a user, wherein the needle arrangement is configured to be brought into fluid communication with the dispensing side of the pouch; a pressure member; and an energy storage member coupled to the pressure member, the energy storage member being loaded in an initial state of the drug delivery device and being configured for squeezing the drug out of the chamber of the pouch by the pressure member during a dispensing operation of the drug delivery device.

### CROSS-REFERENCE TO RELATED APPLICATIONS

The present application is the national stage entry of International Patent Application No. PCT/EP2024/065442, filed on Jun. 5, 2024, and claims priority to European Application No. 23315233.9, filed on Jun. 5, 2023, the disclosures of which are incorporated herein by reference.

### TECHNICAL FIELD

The present disclosure relates to a drug delivery device, in particular a very compact and wieldy drug delivery device.

### BACKGROUND

In drug delivery devices, drug is often delivered to a user via a needle which pierces the skin of the user (or patient). The drug may be accommodated within a drug container of the drug delivery device, e.g. within a syringe arranged within the drug delivery device. Conventional drug delivery devices comprising syringes and an associated drive mechanism have a shape basically corresponding to the shape of the syringe. In particular, conventional drug delivery devices comprising syringes have an elongated cylindrical shape, wherein an axis of the drug delivery device may correspond to an axis of the needle. Such a drug delivery device may often be referred to as pen-type device. The cylindrical form may be hard to handle, in particular if the user has some motoric impairment.

### SUMMARY

It is an object of the present disclosure to facilitate improvements associated with drug delivery devices, particularly with respect to size, shape and operability.

This object is achieved by the disclosed subject-matter, for example by the subject-matter defined in the appended independent claim. Advantageous refinements and developments are subject to dependent claims and/or set forth in the description below.

One aspect of the present disclosure relates to a drug delivery device, comprising: a housing for receiving a pouch within the housing, the pouch having collapsible walls enclosing a chamber, a dispensing side, and a remote side facing away from the dispensing side, and accommodating a drug within the chamber; a needle arrangement comprising at least one needle being configured to pierce the skin of a user, wherein the needle arrangement is configured to be brought into fluid communication with the dispensing side of the pouch; a pressure member; and an energy storage member coupled to the pressure member, the energy storage member being loaded in an initial state of the drug delivery device and being configured for squeezing the drug out of the chamber of the pouch by the pressure member during a dispensing operation of the drug delivery device.

The pouch may be arranged within the housing. The pouch and the needle arrangement enable to design the housing of the drug delivery device very compact, in particular very flat, and wieldy. This contributes to that the drug delivery device is easy to handle and to operate.

The pressure member is configured for transferring the energy released by the energy storage member to the pouch during the dispensing operation. The pressure member may be arranged on the remote side of the pouch. The pressure member may be used as a bearing, stopper or plunger for squeezing the drug out of the pouch. In this context, the pressure member may be arranged such that it is translatory movable relative to the housing, in particular in a dispensing direction towards the pouch. Alternatively, the pressure member may work with air pressure, wherein the air pressure is used to squeeze the drug out of the pouch. For example, the pressure member may be or may comprise a pressure chamber, wherein an over- or under-pressure within the pressure chamber may be generated by the energy storage member and wherein the pouch is coupled to the pressure chamber such that the pressure within the pressure chamber may be used for squeezing the pouch. For example, the pressure chamber has an opening, which is sealingly covered by a wall of the pouch.

The energy storage member may be a spring. The energy storage member may be preloaded and/or biased in an initial state of the drug delivery device. The energy storage member may be released for initiating the dispensing operation. If the energy storage member is released, the energy storage member may drive the pressure member in the dispensing direction towards the remote side of the pouch. In one embodiment, the energy storage member is a conventional spring, which is coupled to the pressure member, and which is loaded and locked in the initial state of the drug delivery device. The spring and the pressure member may be attached to and/or may be arranged within a drive portion. The drive portion may be a part of the housing or may be coupled to the housing. For example, the drive portion comprises a drive portion recess accompanying the spring. In case of the spring, the energy storage member may be pre-loaded by biasing the spring within the drive portion. The energy storage member may allow for a given activation dynamic.

The drug delivery device may be a fully functional drug delivery device. The drug may be a medicament. The drug delivery device may be an autoinjector. In an autoinjector, the energy for the drug delivery operation may be prestored in the energy storage member. That is to say, the user does not have to provide the energy for the drug delivery operation, e.g. when preparing the drug delivery device for use. Rather, this energy may be preloaded into the system by the manufacturer. For example, a drive spring, e.g. a spiral spring or flat spiral spring, may be pre-stressed or pre-biased to provide the energy for the drug delivery operation.

In one embodiment, a distance from the dispensing side to the remote side of the pouch is smaller than a distance from one end of the dispensing side or the remote side to another end of the dispensing side or, respectively, the remote side. The distance from the dispensing side to the remote side may be referred to as height of the pouch. The distance from one end of the dispensing side or the remote side to another end of the dispensing side or, respectively, the remote side may be referred to as length of the pouch. Thus, the height of the pouch, that is the distance from the dispensing side to the remote side of the pouch, is smaller than the length of the pouch, that is the distance from one end of the dispensing side or the remote side to another end of the dispensing side or, respectively, the remote side. So, the pouch is flat. That the length of the pouch is larger than the height of the pouch enables to provide a large surface for piercing the pouch and thereby to pierce the pouch with more than one needle at a time. The flat pouch needs less space in the dispensing direction than a conventional drug container, e.g. a syringe. The pouch may be comprised by the drug delivery device.

In one embodiment, the needle arrangement is configured to pierce the dispensing side of the pouch at a remote side of the needle arrangement and the skin of a user at a dispensing side of the needle arrangement such that the needle arrangement communicates with the chamber and guides the drug under the skin during the dispensing operation. This enables to use a completely sealed pouch, which is pierced during the dispensing operation only.

In one embodiment, the needle arrangement comprises at least two needles. This is particularly advantageous, if the needles pierce the long dispensing side of the pouch, because then the drug may be injected via several needles at a time and/or over a large injection area. This contributes to a large absorption capacity. The needles are configured for injecting the drug into an injection site at their dispensing ends and for being communicatively coupled to the pouch at their remote sides. The needles may extend in a direction parallel to the dispensing direction. The needle arrangement and thereby the needles may be configured for piercing the skin of the user. The needles may be couplable to the pouch or may permanently coupled to the pouch such that the needles may communicate with the chamber of the drug container. A distance between the needles may be far enough so that they do not interfere with each other when administering the medication and/or at least less than the outer dimensions of the pouch. For example, the distance between the needles or further needles may be between 0.1 mm and 30 mm, e.g. between 1 mm and 10 mm. A diameter of the needles may for example correspond to 27G TW or 29 G (G: Gauge). However, smaller or larger needles might be applied as well.

The drug delivery device may comprise a removable multi needle shield being configured to cover and as such protect all of the needles simultaneously before using the drug delivery device.

In one embodiment, the needles are formed and arranged such that at least one of the needles provides a first injection depth and that at least one other of the needles provides a second injection depth. The first injection depth may be different from the second injection depth. For example, the first injection depth may be less than the second injection depth. This enables to inject the drug into different tissue layers and/or the same tissue layer, e.g. subcutaneously, but with different injection depths simultaneously. This may contribute to a high absorption capacity of the drug, e.g. into the skin or into the tissue.

In one embodiment, the needle arrangement comprises at least three needles, wherein the needles providing the different injections depths are alternately arranged. This contributes to a homogeneous injection of the drug and thereby to a very high absorption capacity of the drug into the skin.

In one embodiment, at least two of the needles have different lengths. This enables to provide the above different injection depths in an easy way.

In one embodiment, the needles are arranged as a linear array or as a planar array. The arrays are linear or planar when seen from the top or bottom and/or in the dispensing direction of the drug delivery device. The linear array may be straight or curved. The linear array may enable to use a narrow and/or tube-shaped pouch, wherein “narrow” may refer to that side of the pouch which is perpendicular to its height and perpendicular to its length. The planar array may be rectangular or circular. The planar array may enable to use a broad, and rectangular or circular pouch, wherein “broad” may refer to that side of the pouch which is perpendicular to its height and perpendicular to its length.

In one embodiment, the drug delivery device comprises a needle sleeve for protecting the needle arrangement in the initial state, the needle sleeve being operatively coupled to the energy storage member and being configured such that a release of the energy storage member is prevented as long as the needle sleeve protects the needle arrangement and that the energy storage member is released, when the needle sleeve exposes the needle arrangement. The needle sleeve may be configured for protecting the needle arrangement in a final state after the use of the drug delivery device also. The needle sleeve may be provided to cover the needles in the initial state before the drug delivery device is arranged on the injection site and in the final state after removing the drug delivery device from the injection site. So, the needle sleeve may be provided to cover the needles before the needle pierce the skin and/or after the needles have been removed from the skin, e.g. after completion of the drug delivery operation. Before the drug delivery operation is commenced, the needle sleeve may protrude from the housing, e.g. to cover the tips of the needles (such as by axially extending beyond the tip of needle, e.g. at least with a bearing surface of the needle sleeve). For the drug delivery operation, the needle sleeve may be displaced relative to the housing. After completion of the drug delivery operation, the needle sleeve may be moved relative to the housing, e.g. to cover the tip of needle and/or into a third position relative to the housing. The needle sleeve may be at least partly movable into the housing, if the drug delivery device is arranged on an injection site and is pressed against the injection site. The needle sleeve may expose the needle, if it is at least partly moved into the housing.

The drug delivery device may comprise a needle sleeve spring which is coupled to the needle sleeve and the housing such that the needle sleeve spring is loaded, if the drug delivery device is arranged on the injection site, and that the needle sleeve is pushed out of the housing by the needle sleeve spring, if the drug delivery device is removed from the injection site. The needle sleeve spring may be operatively couplable to or coupled to the needle sleeve in order to move the needle sleeve, e.g. in the dispensing direction relative to the housing. The force of the needle sleeve spring may have to be overcome in order to move the needle sleeve into the housing. In a final position, e.g. after the drug delivery operation has been completed and the drug delivery device has been removed from the skin, the needle sleeve may be locked against a further movement with respect to the housing, such as by a locking mechanism. This may contribute to a safe handling of the drug delivery device after its usage by protecting the used needle.

In one embodiment, the drug delivery device comprises a flexible pouch sealing member which separates the needles from the pouch in the initial state and which is configured for being pierced by the needles for initiating the dispensing operation. The pouch sealing member may sealingly and/or sterilely separate a section of the housing in which the pouch is arranged from a section of the housing in which the needles are arranged. The pouch sealing member may be flexible. The pouch sealing member may comprise or may be a foil.

In one embodiment, the drug delivery device comprises a flexible needle sealing member which covers tips of the needles at their dispensing ends in the initial state and which is configured for being pierced by the needles for the dispensing operation. The needle sealing member may sealingly and/or sterilely protect the needles in the initial state. The needle sealing member may be flexible. The needle sealing member may comprise or may be a foil.

In one embodiment, the drug delivery device comprises a drive portion having a drive portion recess for accommodating the pressure member and the energy storage member, the drive portion being arranged on the remote side of the pouch, and a release mechanism being configured for locking the pressure member and/or the energy storage member within the drive portion in the initial state and for releasing the pressure member and/or, respectively, the energy storage member for the dispensing operation. The drive portion may be a part of the housing or may be coupled to the housing. The drive portion may protrude from a top of the housing, the top facing away from the needle arrangement. The energy storage member may be arranged between the pressure member and a ceiling of the drive portion. The energy storage member, e.g. the spring, may be biased in the initial state. The drive portion recess may be closed at the top of the drive portion and may be open towards the dispensing side.

In one embodiment, the release mechanism comprises a first release member at the drive portion, the first release member being configured to retain the pressure member in the initial state and to release the pressure member upon being activated, and a second release member at the needle sleeve, the second release member being configured for activating the first release member for the dispensing operation. The first release member may be configured for retaining the pressure member against a force applied by the energy storage member in the initial state. The drive portion may comprise the first release member. The first release member may be an integral component of the drive portion. The needle sleeve may comprise the second release member. The second release member may be an integral component of the needle sleeve.

In one embodiment, the first release member comprises an inner shoulder of the drive portion, the inner shoulder extending from an inner wall of the drive portion recess radially inwardly and being configured to retain the pressure member in the initial state. For example, an opening of the drive portion recess may be a little bit smaller than a diameter of the pressure member because of the inner shoulder such that the pressure member may not be moved outside of the drive portion recess in the initial state. However, the walls of the drive portion may have such a flexibility that the walls may be sufficiently pulled outwardly that the opening gets larger than the diameter of the pressure member and that the pressure member may be moved outside of the drive portion by the energy storage member. The first release member may also comprise an outer shoulder of the drive portion, the outer shoulder extending radially outwardly and being configured for being pushed radially outwardly by the second release member thereby pulling the inner shoulder radially outwardly to release the pressure member for initiating the dispensing operation.

In one embodiment, the first or second release member comprises a chamfer and the other of the first and second release member comprises a pin, wherein the chamfer and the pin are formed and arranged such that the first release member is pressed outwards in the radial direction by the second release member when the needle sleeve is moved into the housing. The first release member may be arranged at the drive portion and the second release member may be arranged at the needle sleeve. For example, the first release member comprises the pin and the second release member comprises the chamfer.

We note that features described above and below in conjunction with different embodiments or aspects can be combined with one another, even if such a combination is not explicitly disclosed herein above or below. Further features, advantages and expediencies of the disclosure and, particularly, of the proposed concepts will become apparent from the following description of the exemplary embodiments in conjunction with the drawings.

### BRIEF DESCRIPTION OF THE DRAWINGS

FIG. 1 illustrates a cross-sectional side view of an interior of an exemplary embodiment of a drug delivery device in a first state.

FIG. 2 illustrates cross-sectional side view of the interior of the drug delivery device of FIG. 1 in a second state.

FIG. 3 illustrates cross-sectional side view of the interior of the drug delivery device of FIG. 1 in a third state.

FIG. 4 illustrates cross-sectional side view of the interior of the drug delivery device of FIG. 1 in a fourth state.

FIG. 5 illustrates cross-sectional side view of the interior of the drug delivery device of FIG. 1 in a fifth state.

FIG. 6 illustrates a bottom view of an exemplary embodiment of a needle arrangement.

FIG. 7 illustrates a bottom view of an exemplary embodiment of a needle arrangement.

FIG. 8 illustrates a cross-sectional side view of an interior of an exemplary embodiment of a drug delivery device in a first state.

FIG. 9 illustrates a cross-sectional side view of the interior of the drug delivery device of FIG. 8 in a second state.

FIG. 10 illustrates an exemplary embodiment of a needle sleeve in a first state.

FIG. 11 illustrates the needle sleeve of FIG. 10 in a second state.

FIG. 12 illustrates the needle sleeve of FIG. 10 in a third state.

FIG. 13 illustrates the needle sleeve of FIG. 10 in a fourth state.

FIG. 14 illustrates the needle sleeve of FIG. 10 in a fifth state.

FIG. 15 illustrates the needle sleeve of FIG. 10 in a sixth state.

FIG. 16 illustrates an expanded structural formula, molecular formula, and molecular weight of fitusiran.

Identical elements, elements of the same kind and identically or similarly acting elements may be provided with the same reference numerals in the drawings.

### DETAILED DESCRIPTION

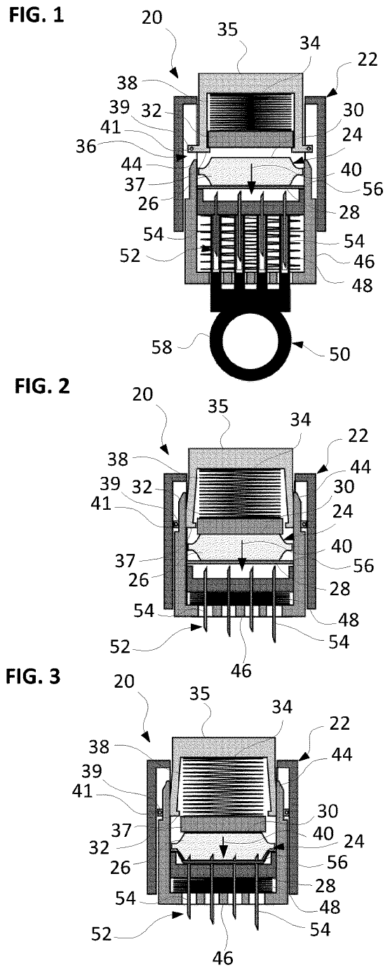

FIG. 1 illustrates a cross-sectional side view of an interior of an exemplary embodiment of a drug delivery device 20 in a first state. In particular, FIG. 1 shows the drug delivery device 20 in an initial or as delivered state. The drug delivery device 20 comprises a housing 22, a drive mechanism, a needle arrangement 52, a needle sleeve 46, and a release mechanism. The housing 22 is provided to accommodate and/or accommodates a pouch 24.

The drug delivery device 20 may be an autoinjector. The energy for driving the drug delivery operation in an autoinjector may be provided by components integral to the drug delivery device 20 and does not have to be loaded into the drug delivery device 20 by the user during the operation as is the case in many spring driven pen-type variable dose injectors, where, usually, the energy is loaded into a spring by the user during a dose setting procedure. The drug delivery device 20 expediently is a single shot device, i.e. it is provided to dispense only one dose. The drug delivery device 20 may be a disposable drug delivery device 20, that is to say a drug delivery device 20 which is disposed of after its use. The pouch 24 and/or the needle arrangement 52 may be secured within the drug delivery device 20, e.g. within the housing 22. So, the user may have to perform the movement for piercing the skin with the needle arrangement 52 by placing the drug delivery device 20 on the skin.

The pouch 24 may be arranged within a cavity of the housing 22. The pouch 24 comprises a dispensing side 28 and a remote side 30 opposite the dispensing side 28. A distance from the dispensing side 28 to the remote side 30 of the pouch 24 may be smaller than a distance from one end of the dispensing side 28 to another end of the dispensing side 28. Alternatively or additionally, the distance from the dispensing side 28 to the remote side 30 of the pouch 24 is smaller than a distance from one end of the remote side 30 to another end of the remote side 30. The distance from the dispensing side 28 to the remote side 30 may be referred to as height of the pouch 24. The distance from one end of the dispensing side 28 or the remote side 30 to another end of the dispensing side 28 or, respectively, the remote side 30 may be referred to as length of the pouch 24. So, the pouch 24 may be flat. The pouch 24 comprises a chamber 26. A drug, i.e. a medicament, e.g. liquid medicament, may be arranged within the chamber 26. The pouch 24 may comprise flexible walls. The walls of the pouch 24 may be collapsible. For example, the walls are made of foils, e.g. an upper foil at the remote side 30 and a lower foil at the dispensing side 28. The chamber 26 is fluid-tight closed. For example, the upper and lower foil are sealingly coupled to each other at a seam of the pouch 24 at the peripheral regions of the foils. The pouch 24 may be squeezed from the remote side 30 towards the dispensing side 28 to dispense the drug retained within the chamber 26.

The drive mechanism provided to drive a drug delivery operation is expediently provided in the housing 22. In particular, the drive mechanism may be provided to squeeze the drug out of the pouch 24. The drive mechanism comprises a pressure member 32 for pressuring the pouch 24 from the remote side 30 in a dispensing direction towards the dispensing side 30, an energy storage member 34 coupled to the pressure member 32 for moving the pressure member 32, and a drive portion 35 for accommodating the pressure member 32 and the energy storage member 34. The pressure member 32 may be movable in the dispensing direction 40 towards the dispensing side 28. If the pressure member 32 is moved in the dispensing direction 40, firstly the pressure member 32 hits the remote side 30 of the pouch 24 and then presses the remote side 30 towards the dispensing side 28 and thereby squeezes the pouch 24.

The drive portion 35 may be cup-shaped and/or may have a drive portion recess 38 for accommodating the pressure member 32 and the energy storage member 34. An opening of the cup-shaped drive portion 35 may face the pouch 24. So, in FIG. 1, the cup-shaped drive portion 35 is orientated upside down. The drive portion 35 is arranged on the remote side 30 of the pouch 24. The drive portion 35 may be a part of the housing 22 or may be coupled to the housing 22. The drive portion 35 may protrude from a top of the housing 22, the top facing away from the pouch 24. The energy storage member 34 may be arranged between the pressure member 32 and an inner ceiling of the drive portion recess 38. The drive portion recess 38 may be closed at the top of the drive portion 35 and may be open towards the dispensing side 28.

The energy storage member 34 may be a conventional spring, which is coupled to the pressure member 32. The energy storage member 34, e.g. the spring, may be is loaded and locked in the initial state of the drug delivery device 20. In the initial and loaded state, energy is stored in the loaded energy storage member 34, i.e. the spring is biased. If the energy storage member 34 is released, the energy stored in the energy storage member 34 is released and transferred to the pressure member 32 such that the drug is squeezed out of the pouch, e.g. by the pressure member 32. Other potential drive energy sources different from a spring may comprise an electrical power cell or battery for driving the pressure member 32 by a motor or a reservoir suitable to provide gas pressure, where the gas pressure can be used to drive the drug delivery operation. Further, this gas pressure may be used to squeeze the pouch 24 directly, without any pressure transferring element. For example, the pressure member 32 may be a gas chamber having an opening, wherein the remote side 30 or the dispensing side 28 of the pouch 24 may sealingly cover the opening. Then, depending on which side of the pouch 24 covers the opening and depending on whether the opening is covered by the corresponding side from inside or from outside, an under-pressure or, respectively, an over-pressure within the pressure member 32 may lead to the squeezing of the pouch 24.

The pressure member 32 may be coupled to the energy storage member 34 such that a locking of the pressure member 32 corresponds to a locking of the energy storage member 34. So, if the pressure member 32 is held in its initial state, the energy storage member 34 may be held in its initial state also. To hold the pressure member 32 in its initial state and to unlock the pressure member 32 for the dispensing operation, the drug delivery device 20 may comprise a release mechanism 36.

The release mechanism 36 may be configured for locking the pressure member 32 and/or the energy storage member 34 within the drive portion 35 in the initial state and for releasing the pressure member 32 and/or, respectively, the energy storage member 34 for the dispensing operation. The release mechanism 36 for locking the energy storage member 34 in the initial state may be operatively coupled to or may comprise a part of a needle sleeve 46 such that the release mechanism 36 releases the pressure member 32 and/or the energy storage member 34 upon moving the needle sleeve 46 into the housing 22.

The needle arrangement 52 is configured to pierce the dispensing side 28 of the pouch 24 at a remote side of the needle arrangement 52 and the skin of a user at a dispensing side of the needle arrangement 52 such that the needle arrangement 52 communicates with the chamber 26 and guides the drug under the skin during the dispensing operation. The dispensing side of the needle arrangement faces away from the pouch 24 and the remote side of the needle arrangement 52 faces the pouch 24. The needle arrangement 52 comprises at least one, preferably two or more than two needles 54.

The needles 54 may extend in a direction parallel to the dispensing direction 40. In the initial state of the drug delivery device 20, the needles 54 are separated from the fluid-tightly closed pouch 24. A fluid communication between an interior of the pouch 24 and the needles 54 may be only established during operation of the drug delivery device 20. The needles 54 may be formed and arranged such that at least one of the needles 54 provides a first injection depth and that at least one other of the needles 54 provides a second injection depth. The first injection depth may be different from the second injection depth. For example, the first injection depth may be less than the second injection depth. For example, the needle arrangement 52 comprises at least three, e.g. four, needles 54, wherein the needles 54 providing the different injections depths are alternately arranged. Alternatively or additionally, at least two of the needles 54 may have different lengths in order to provide the different injection depths.

The needle arrangement 52, in particular the needles 54, may be protected by a multi needle shield 50 prior to the use of the drug delivery device 20. The multi needle shield 50 may cover the needles 54 until it is removed by hand. The multi needle shield 50 may comprise a gripping ring 58. The gripping ring 58 may provide a comfortable gripping structure for gripping and removing the multi needle shield 50.

The drug delivery device 20 may comprises a flexible pouch sealing member 56. The pouch sealing member 56 may sealingly and/or sterilely separate a section of the housing 22 in which the pouch 24 is arranged from a section of the housing 22 in which the needles 54 are arranged. The pouch sealing member 56 may be flexible. The pouch sealing member 56 may comprise or may be a foil. The flexible pouch sealing member 56 may separate the needles 54 against the pouch 24 in the initial state. The flexible pouch sealing member 56 may be configured for being pierced by the needles 54 for initiating the dispensing operation.

The needle sleeve 46 may be arranged for protecting the needle arrangement 52, in particular the needles 54 of the drug delivery device 20. The needle sleeve 46 may be provided to extend beyond a tip of the needles 54, which may protrude from the bottom of the housing 22 before the drug delivery operation is commenced. The needle sleeve 46 may protrude from the housing 22 in the initial state. The needle sleeve 46 may be movably arranged within the housing 22. In particular, the needle sleeve 46 may be moved parallel to the dispensing direction 40 and as such may be further introduced into the housing 22. The needle sleeve 46 may be movable relative to the housing 22 from an initial position or first position in the initial state to a second position or trigger position. During this movement, e.g. before the needle sleeve 46 reaches the second position, the needles 54 may pierce the skin of the user. The needle sleeve 46 may be pushed into the housing 22 when the drug delivery device 20 is arranged on the injection site, e.g. the skin of the user. In particular, if the drug delivery device 20 is positioned on the skin with a bottom surface of the needle sleeve 46 touching the skin, the needle sleeve 46 is pushed into the housing 22. If the needle sleeve 46 is pushed into the housing 22, the needles 54 is exposed and may pierce the skin. The bottom surface of the needle sleeve 46 may provide at least a part of a bearing surface of the drug delivery device 20. The bearing surface is meant for being in contact with the injection site during the dispensing operation, the bearing surface facing away from the drive portion 35.

The drive portion 35 and the needle sleeve 46 each may comprise a part of the release mechanism 36. The release mechanism 36 may comprise a first release member at the drive portion 35. The release mechanism 36 may comprise a second release member at the needle sleeve 46. The first release member may be configured to retain the pressure member 32 and/or the energy storage member 34 in the initial state and to release the pressure member 23 and/or, respectively, the energy storage member 34, upon being activated. The second release member may be configured for activating the first release member for the dispensing operation.

The drive portion 35 may comprise the first release member. The first release member may be an integral component of the drive portion, i.e. the first release member and the drive portion 35 may be a one-piece component. The needle sleeve 46 may comprise the second release member. The second release member may be an integral component of the needle sleeve, i.e. the second release member and the needle sleeve 46 may be a one-piece component.

The first release member may be configured for retaining the pressure member 32 against a force applied by the energy storage member 35 in the initial state. The first release member may comprise an inner shoulder 37 of the drive portion 35. The inner shoulder 37 may extend from an inner wall of the drive portion recess 38 radially inwardly. The inner shoulder 37 may extend over the whole circumference of the opening or may only be formed and/or arranged within one, two or more angular sections of the circumference of the opening. The inner shoulder 37 may be configured to retain the pressure member 32 in the initial state. For example, an opening of the drive portion recess 38 may be a little bit smaller, e.g. narrower, than a diameter of the pressure member 32 because of the inner shoulder 37 such that the pressure member 32 may not be moved outside of the drive portion recess 38 in the initial state. However, the walls of the drive portion 35 may have such a flexibility that the walls may be sufficiently pulled outwardly that the opening gets larger than the diameter of the pressure member 32 and that the pressure member 32 may be moved outside of the drive portion 35 by the energy storage member 34. The first release member may also comprise an outer shoulder 39 of the drive portion 35, the outer shoulder 39 extending radially outwardly and being configured for being pushed radially outwardly by the second release member thereby pulling the inner shoulder 37 radially outwardly to release the pressure member 32 for initiating the dispensing operation. The second release member may comprise a chamfer 44 at the needle sleeve 46. The first release member may comprise a pin 41, wherein the chamfer 44 and the pin 41 are formed and arranged such that the pin 41 is pressed outwardly by the chamfer 44 when the needle sleeve 46 is moved into the housing 22. Alternatively, the first release member may comprise the chamfer 44, wherein the chamfer 44 is arranged at the drive portion 35, and the second release member may comprise the pin 41, wherein the pin 41 may be arranged at the needle sleeve 46. In this alternative embodiment, the chamfer 44 and the pin 41 are formed and arranged such that the chamfer 44 is pressed outwardly by the pin 41 when the needle sleeve 46 is moved into the housing 22.

The drug delivery device 20 may comprise a needle sleeve spring 48 which is coupled to the needle sleeve 46 and the housing 22. The needle sleeve spring 48 may be operatively couplable to or coupled to the needle sleeve 46 in order to move the needle sleeve 46, e.g. in the dispensing direction relative to the housing 22. The needle sleeve spring 48 may be loaded, if the drug delivery device 20 is arranged on the injection site and if the needle sleeve 46 is pressed into the housing 22. The force of the needle sleeve spring 48 may have to be overcome in order to move the needle sleeve 46 into the housing 22. If the drug delivery device 20 is removed from the injection site, the needle sleeve 46 may be pushed out of the housing 22 by the needle sleeve spring 48. In a final position, e.g. after the drug delivery operation has been completed and the drug delivery device 20 has been removed from the skin, the needle sleeve 46 may be locked against a further movement with respect to the housing 22, such as by a locking mechanism, as explained below with respect to FIGS. 10 to 15.

The needle sleeve 46 may serve as a trigger member of the drug delivery device 20. The needle sleeve 46 as trigger member, when displaced from the initial or first position depicted in FIG. 1 to a second or trigger position depicted in FIG. 2, may automatically initialize the drug delivery operation, preferably when it is in the second position.

FIG. 2 illustrates cross-sectional side view of the interior of the drug delivery device 20 of FIG. 1 in a second state. In the second state, the needle shield 50 has been removed and the needle sleeve 46 is pushed into the housing 22 in its second position, e.g. by arranging the drug delivery device 20 on the injection site and by pressing the housing 22 towards the injection site. The needle sleeve 46, when moved from the first position to the second position and expediently when in the second position, may initiate the drug delivery operation via acting on the first release member of the release mechanism 36. Operating the first release member to initiate the drug delivery operation may only be possible when the needle sleeve 46 is in the second position. In particular, if the needle sleeve 46 is pushed into the housing 22, the second release member, e.g. the chamfer 44, may act on the first release member, in particular the pin 41 at the outer shoulder 39 of the drive portion 35 such that the flexible walls of the drive portion 35 together with the inner shoulder 37 are pulled radially outwardly thereby releasing the locked pressure member 32 and thereby the biased energy storage member 34. Then, the energy storage member 34 pushes the pressure member 32 in the dispensing direction 40 towards the pouch 24. So, the pressure member 32 may be moved only, if the drug delivery device 20 is positioned on the skin and the needle sleeve 46 exposes the needles 54. In the second state, the pressure member 32 touches the remote side 30 of the pouch 24, but the pouch 24 is not yet squeezed.

FIG. 3 illustrates cross-sectional side view of the interior of the drug delivery device 20 of FIG. 1 in a third state. Just before the third state, the pouch sealing member 56 may be deformed only as long as the pouch 24 is not yet pierced, because the pressure of the pressure member 32 is transferred to the flexible sealing member 56 by the pressurized but still closed pouch 24. In the third state, the energy storage member 34 presses the pouch 24 against the pouch sealing member 56 and the needle arrangement 52 such that the pouch sealing member 56 is deformed and that the remote ends of the needles 54 pierce the dispensing side 28 of the pouch 24. So, in the third state, the dispensing side 28 of the pouch 24 and the pouch sealing member 56 are pierced, but the pouch 24 is not yet squeezed further.

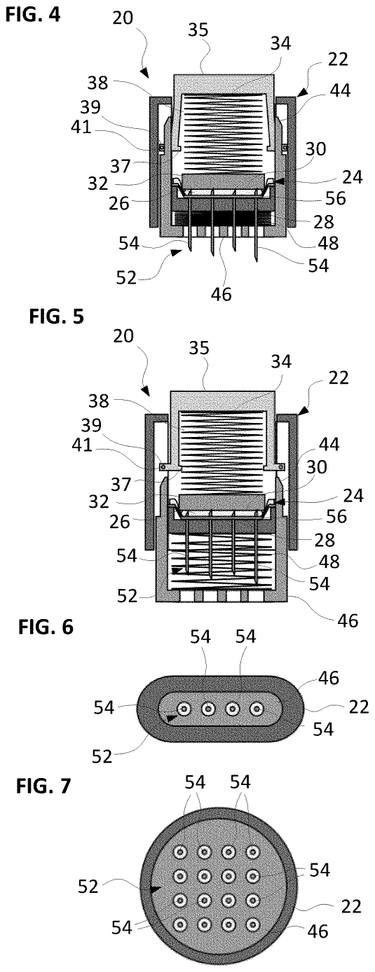

FIG. 4 illustrates cross-sectional side view of the interior of the drug delivery device 20 of FIG. 1 in a fourth state. In the fourth state, the pouch 24 is squeezed by the pressure member 32 in the dispensing direction and the drug is pressed out of the chamber 26 through the needles 54 into the skin of the user (not shown).

FIG. 5 illustrates cross-sectional side view of the interior of the drug delivery device of FIG. 1 in a fifth state. In the fifth state, the drug delivery device 20 is removed from the skin, wherein the needle sleeve 46 is pushed out of the housing 22 by the needle sleeve spring 48. Optionally, in the fifth state, the needle sleeve 56 is locked such that it may not be moved towards the housing 22 anymore, as explained below with respect to FIGS. 10 to 15.

FIG. 6 illustrates a bottom view of an exemplary embodiment of a needle arrangement 52, e.g. the above needle arrangement 52. The needles of the needle arrangement 52 are linearly arranged, in particular as a straight line.

FIG. 7 illustrates a bottom view of an exemplary embodiment of a needle arrangement 52, e.g. the above needle arrangement 52. The needles 54 of the needle arrangement 52 are arranged as an array, in particular as a rectangular, e.g. a quadratic, array. Alternatively, the array may have any polygonal or circular shape.

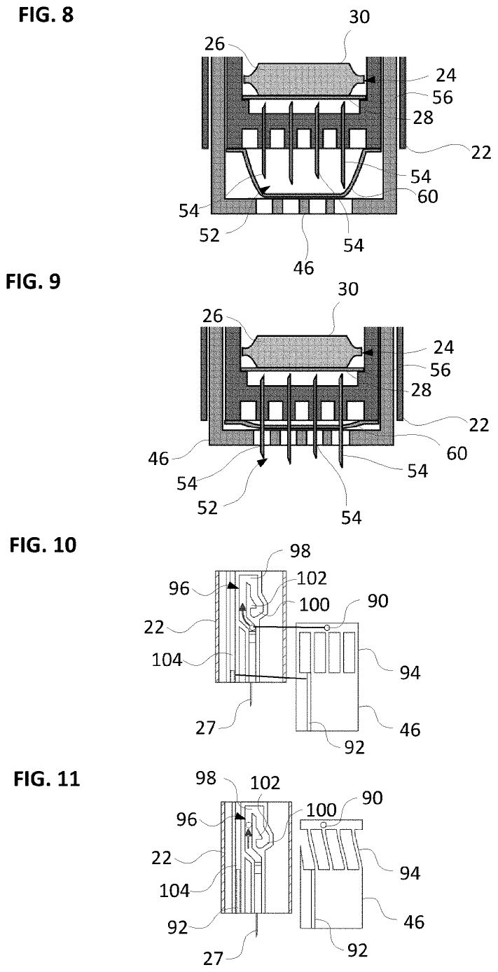

FIG. 8 illustrates a cross-sectional side view of an interior of an exemplary embodiment of a drug delivery device 20 in a first state. The first state shown in FIG. 8 may correspond to the above first state shown in FIG. 1. The drug delivery device 20 may widely correspond to the above drug delivery device 20. Therefore, only those features of the drug delivery device 20 are explained in the following, in which the drug delivery device 20 shown in FIG. 8 differs from the above drug delivery device 20. The drug delivery device 20 may comprise a flexible needle sealing member 60. The needle sealing member 60 may sealingly and/or sterilely separates a section of the housing 22 and/or the needle sleeve 46 in which the needle arrangement 52 is arranged from through-recesses within the needle sleeve 46 at the bearing surface of the needle sleeve 46. The needle sealing member 60 may be flexible. The needle sealing member 60 may comprise or may be a foil. The flexible needle sealing member 60 may separate the tips of the needles 54 against the environment in the initial state. The flexible needle sealing member 60 may be configured for being pierced by the needles 54 for initiating the dispensing operation.

FIG. 9 illustrates a cross-sectional side view of the interior of the drug delivery device 20 of FIG. 8 in a second state. In the second state, the flexible needle sealing member 60 may be pierced by the needles 54. The second state shown in FIG. 9 may correspond to the above second state shown in FIG. 2.

FIG. 10 illustrates an exemplary embodiment of the needle sleeve 46 and the housing 22 in a first state. In particular, FIG. 10 illustrates a side view of the needle sleeve 46 and a cutaway side view of the housing 22 in the first state. In the first state of the needle sleeve 46, the drug delivery device 20 is not yet arranged on the skin of the user and the needle sleeve 46 protects the needles 54.

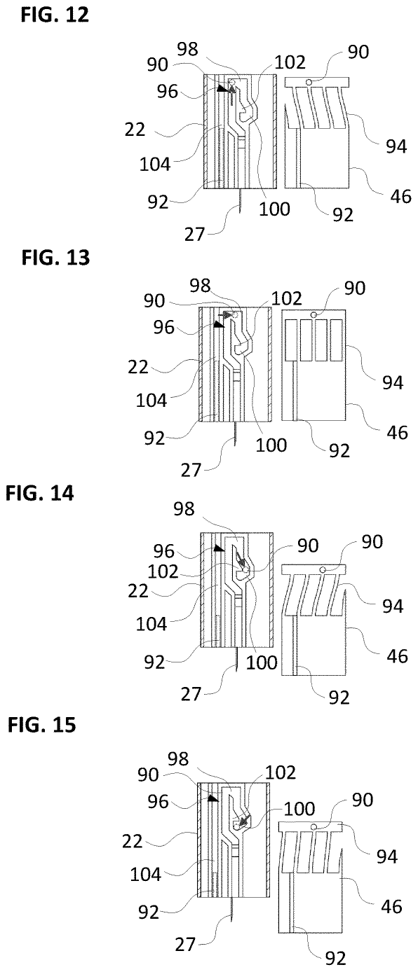

The needle sleeve 46 comprises a guide pin 90, a torsion protection 92, and flexible bars 94. The guide pin 90 protrudes from an outer wall of the needle sleeve 46 outwardly. The flexible bars 94 are separated by through-recesses extending through the wall of the needle sleeve 46. So, the flexible bars 94 and the body of the needle sleeve 46 may be made of one piece. The torsion protection 92 may comprise a bar extending vertically in FIG. 12.

The housing 22 comprises a first channel 96 and a second channel 104. The first channel 96 extends firstly with a slide inclination against the vertical direction and then basically verticality towards a bent 98 of the first channel 96 and then back towards a dead end 100 of the first channel 96. The dead end 100 is separated from the rest of the first channel 96 by a barb 102. In the first state of the needle sleeve 46, the guide pin 90 is arranged in a part of the first channel 96 below the dead end 100 and at the beginning of the inclination of the first channel 96.

The torsion protection 92 of the needle sleeve 46 is arranged within the second channel 104 and is guided by the second channel 104 during the movement of the needle sleeve 46 relative to the housing 22. The second channel 104 is straight and parallel to the axis 45 and as such parallel to the moving direction of the needle sleeve 46. The torsion protection 92 within the second channel 104 serves as a protection of the needle sleeve 46 against a rotation of the needle sleeve 46.

FIG. 11 illustrates the needle sleeve 46 and the housing 22 of FIG. 10 in a second state. In the second state of the needle sleeve 46, the needle sleeve 46 may be partly arranged within the housing 22, e.g. because of the drug delivery device 20 being partly arranged on the skin of the user. In the second state of the needle sleeve 46, the guide pin 90 is moved within the first channel 96 towards the bent 98. When the guide pin 90 passes the inclination of the first channel 96, an upper portion of the needle sleeve 46 is moved perpendicular to the moving direction of the needle sleeve 46 and the flexible bars 94 are flexed, because the rest of the needle sleeve 46 is secured against any rotation by the torsion protection 92 within the second channel 104. Then, the flexible bars 94 are biased.

FIG. 12 illustrates the needle sleeve 46 and the housing 22 of FIG. 10 in a third state. In the third state of the needle sleeve 46, the needle sleeve 46 is pressed into the housing 22 completely, e.g. because of the user arranging the drug delivery device 20 on his/her skin. So, in the third state of the needle sleeve 46, the needles 54 is exposed by the needle sleeve 46. In this situation, the guide pin 90 has arrived in the bent 98 of the first channel 96 and may be moved perpendicular to the moving direction of the needle sleeve 46 within the bent 98. In the third state of the needle sleeve 46, the biased flexible bars 94 force the guide pin 90 through the bent 98.

FIG. 13 illustrates the needle sleeve 46 and the housing 22 of FIG. 10 in a fourth state. In the fourth state of the needle sleeve 46, the flexible bars 94 are released and the guide pin 90 has moved perpendicular to the moving direction of the needle sleeve 46 within the bent 98.

FIG. 14 illustrates the needle sleeve 46 and the housing 22 of FIG. 10 in a fifth state. In the fifth state of the needle sleeve 46, the drug delivery device 20 may be partly removed from the skin of the user. The guide pin 90 is forced over the barb 102 of the first channel 100 such that the flexible bars 94 are biased again. When the drug delivery device 20 is removed from the skin of the user, the needle sleeve 46 may be pushed out of the housing 22, for example by the needle sleeve spring 48 such that the guide pin 90 is forced over the barb.

FIG. 15 illustrates the needle sleeve 46 and the housing 22 of FIG. 10 in a sixth state. In the sixth state of the needle sleeve 46, the drug delivery device 20 may be completely removed from the skin of the user. The needle sleeve 46 may completely cover the needles 54. The guide pin 90 snaps into the dead end 100 of the first channel 96 such that the needle sleeve 46 is fixedly engaged to the housing 22.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of pharmaceutical formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug reservoir” adapted for use with a drug delivery device. The drug reservoir 101a may be, e.g., a cartridge, syringe, reservoir, or other solid or flexible vessel (bag) configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug reservoir may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrome.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in conjunction with various embodiments of the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Further examples of APIs for the prophylaxis of hemophilia A or B, with or without inhibitors, include an siRNA targeting antithrombin. An example of an siRNA targeting antithrombin is fitusiran. The term “prophylaxis” and “prophylactic treatment” are used interchangeably herein

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, pharmaceutical formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

Fitusiran as the API for the medicament in the device

Fitusiran is a synthetic, chemically modified double-stranded small interfering RNA (siRNA) oligonucleotide covalently linked to a tri-antennary N-acetyl-galactosamine (GalNAc) ligand targeting AT3 mRNA in the liver, thereby suppressing the synthesis of antithrombin. See, e.g., Pasi et al., N Engl J Med. (2017) 377(9):819-28. The nucleosides in each strand of fitusiran are connected through either 3′-5′ phosphodiester or phosphorothioate linkages, thus forming the sugar-phosphate backbone of the oligonucleotide.

The sense strand and the antisense strand contain 21 and 23 nucleotides, respectively. The 3′ end of the sense strand is conjugated to the GalNAc containing moiety (referred to herein as L96) through a phosphodiester linkage. The sense strand contains two consecutive phosphorothioate linkages at its 5′ end. The antisense strand contains four phosphorothioate linkages, two at the 3′ end and two at the 5′ end. The 21 nucleotides of the sense strand hybridize with the complementary 21 nucleotides of the antisense strand, thus forming 21 nucleotide base pairs and a two-base overhang at the 3′-end of the antisense strand. See also U.S. Pat. Nos. 9,127,274, 11,091,759, US2020/0163987A1, and WO 2019/014187, the entire contents each of which are expressly incorporated herein by reference.

The two nucleotide strands of fitusiran are shown below:
sense strand: 5′Gf-ps-Gm-ps-Uf-Um-Af-Am-Cf-Am-Cf-Cf-Af-Um-Uf-Um-Af-Cm-Uf-Um-Cf-Am-Af-L96 3′ (SEQ ID NO:1), and
antisense strand: 5′ Um-ps-Uf-ps-Gm-Af-Am-Gf-Um-Af-Am-Af-Um-Gm-Gm-Uf-Gm-Uf-Um-Af-Am-Cf-Cm-ps-Am-ps-Gm 3′ (SEQ ID NO:2),
wherein
Af=2′-deoxy-2′-fluoroadenosine
Cf=2′-deoxy-2′-fluorocytidine
Gf=2′-deoxy-2′-fluoroguanosine
Uf=2′-deoxy-2′-fluorouridine
Am=2′-O-methyladenosine
Cm=2′-O-methylcytidine
Gm=2′-O-methylguanosine
Um=2′-O-methyluridine
“-” (hyphen)=3′-5′ phosphodiester linkage sodium salt
“-ps-”=3′-5′ phosphorothioate linkage sodium salt
   and wherein L96 has the following formula:

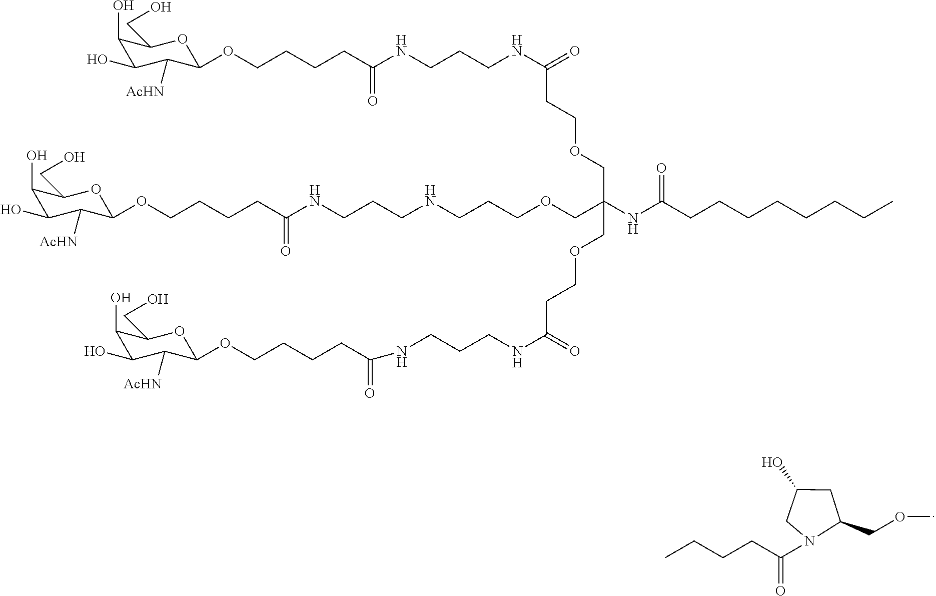

As used herein, the terms 2′-deoxy-2′-fluoroadenosine and 2′-fluoroadenosine may be used interchangeably. As used herein, the terms 2′-deoxy-2′-fluorocytidine and 2′-fluorocytidine may be used interchangeably. As used herein, the terms 2′-deoxy-2′-fluoroguanosine and 2′-fluoroguanosine may be used interchangeably. As used herein, the terms 2′-deoxy-2′-fluorouridine and 2′-fluorouridine may be used interchangeably.

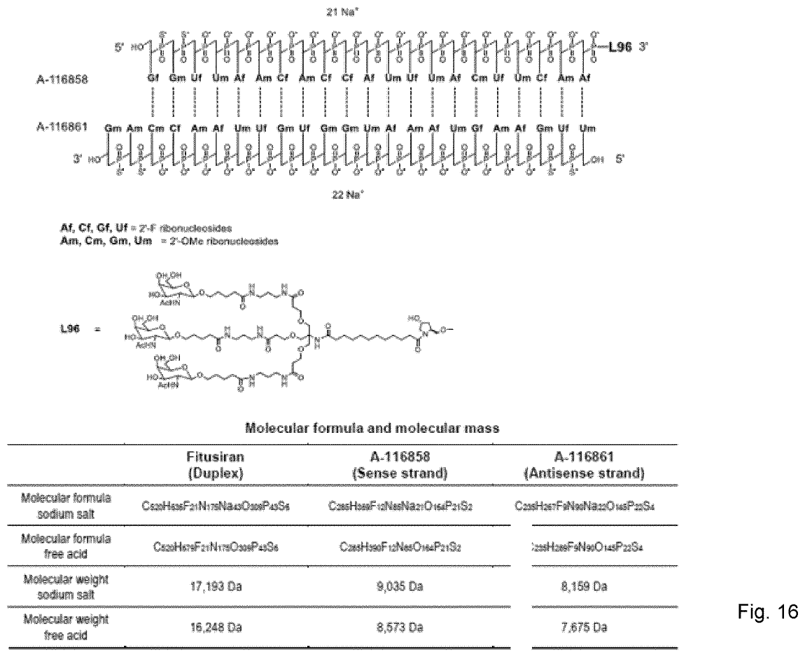

The expanded structural formula, molecular formula, and molecular weight of fitusiran are shown in FIG. 16.

The structure of fitusiran can also be described using the following diagram, wherein the X is O:

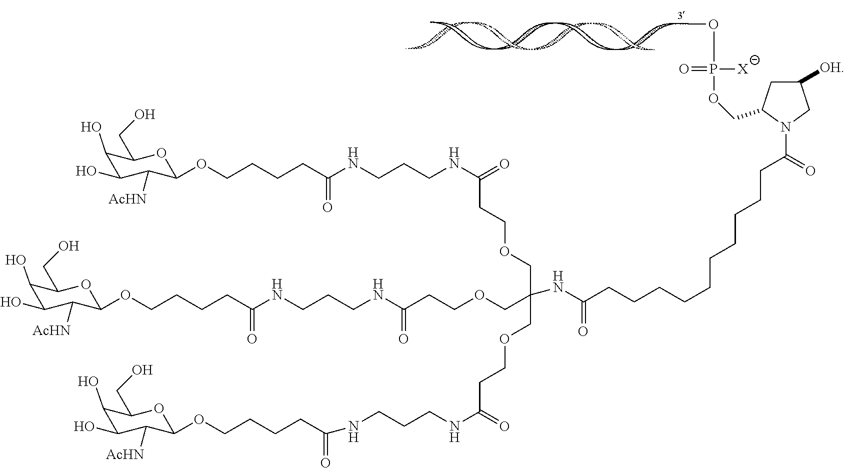

Fitusiran is shown in FIG. 16 in sodium salt form.

In some embodiments, the device delivers fitusiran in an aqueous solution, wherein fitusiran is at a concentration of about 40 to about 200 mg/mL (e.g., about 50 to about 150 mg/mL, about 80 to about 110 mg/mL, or about 90 to about 110 mg/mL). As used herein, values intermediate to recited ranges and values are also intended to be part of this disclosure. In addition, ranges of values using a combination of any of recited values as upper and/or lower limits are intended to be included. In further embodiments, the pharmaceutical formulation comprises fitusiran in an aqueous solution at a concentration of about 40, about 50, about 75, about 100, about 125, about 150, or about 200 mg/mL. In certain embodiments, fitusiran is provided in an aqueous solution at a concentration of about 100 mg/mL.

The term “deliver,” “delivers,” or “delivering” is intended to mean “administer,” “administers,” or “administering.”

Unless specifically stated or otherwise evident from the context, as used herein, the term “approximately” or “about” refers to a value that is within an acceptable error range for a particular value determined by a person of ordinary skill, a portion of which will depend on how the measurement or determination is made. For example, “approximately” or “about” may mean a range of up to 10% (ie, ±10%). Therefore, “approximately” or “about” can be understood as greater than or less than 10%, 9%, 8%, 7%, 6%, 5%, 4%, 3%, 2%, 1%, 0.5%, 0.1%, 0.05%, 0.01%, or 0.001%. When a specific value is provided in this disclosure, unless otherwise stated, the meaning of “approximately” or “about” should be assumed to be within an acceptable error range for that specific value.

While the fitusiran dosage weight described herein refers to the weight of fitusiran free acid (active moiety), administration of fitusiran to patients herein refers to administration of fitusiran sodium (drug substance) provided in a pharmaceutically suitable aqueous solution (e.g., a phosphate-buffered saline at a physiological pH). For example, about 100 mg/mL fitusiran means about 100 mg of fitusiran free acid (equivalent to about 106 mg fitusiran sodium, the drug substance) per mL. Unless otherwise indicated, a fitusiran weight recited in the present disclosure is the weight of fitusiran free acid (the active moiety).

In some embodiments, a pharmaceutical formulation in the device comprises fitusiran in a phosphate-buffered saline. The phosphate concentration in the solution may be about 1 to about 10 mM (e.g., about 2, about 3, about 4, about 5, about 6, about 7, about 8, or about 9 mM), with a pH of about 6.0-8.0. The pharmaceutical formulations herein may include a stabilizing agent such as EDTA. The pharmaceutical formulations may be preservative-free. In some embodiments, the fitusiran pharmaceutical formulation in the device is preservative-free and comprises, consists of, or consists essentially of about 100 mg of fitusiran per mL of an approximately 5 mM phosphate buffered saline (PBS) solution. In some embodiments, the fitusiran pharmaceutical formulation in the device is preservative-free and comprises, consists of, or consists essentially of fitusiran in an approximately 5 mM phosphate buffered saline (PBS) solution. The PBS solution is composed of sodium chloride, dibasic sodium phosphate (heptahydrate), and monobasic sodium phosphate (monohydrate). Sodium hydroxide solution and diluted phosphoric acid may be used to adjust the pH of the pharmaceutical formulation to about 7.0 or about 7.1.

In some embodiments, the fitusiran pharmaceutical formulation in the device for subcutaneous delivery contains fitusiran in a 5 mM phosphate buffered saline having 0.64 mM NaH₂PO₄, 4.36 mM Na₂HPO₄, and 84 mM NaCl at pH 7.0. In certain embodiments, the pharmaceutical formulation of fitusiran solution for subcutaneous delivery is shown in Table 1 below:

TABLE 1
Exemplary Fitusiran Pharmaceutical Formulation

Pharmaceutical Formulation

| | Percentage | Per ml |
|---|---|---|
| Components | [%] | [mg] |
| Fitusiran (active moiety) | 10 | 100 |
| [equivalent to fitusiran sodium] | | [106] |
| Sodium chloride | 0.49 | 4.909 |
| Dibasic sodium phosphate | 0.12 | 1.169 |
| (heptahydrate) | | |
| Monobasic sodium phosphate | <0.01 | 0.0885 |
| (monohydrate) | | |
| Phosphoric acid, concentrated | — | q.s. pH 7.0 |
| Sodium hydroxide | — | q.s. pH 7.0 |
| Water for subcutaneous | q.s. 100 | q.s. 1 mL |
| delivery | | |

*q.s.: quantum satis

In some embodiments, the pharmaceutical formulation of fitusiran solution for subcutaneous delivery with the device can be described as shown in Table 2 below.

TABLE 2
Exemplary Fitusiran Pharmaceutical Formulation

| | Pharmaceutical Formulation |
|---|---|
| Components | (mg) |
| Fitusiran (active moiety) | 100 |
| [equivalent to fitusiran sodium] | [106] |
| NaH₂PO₄*H₂O | 0.0885 |
| Na₂HPO₄*7H₂O | 1.169 |
| NaCl | 4.909 |
| 0.1N NaOH | q.s. |
| 0.1M H₃PO₄ | q.s. |
| Purified water | Ad 1 mL |

In some embodiments, the device may be used to deliver a single dose of fitusiran wherein the single dose comprises about 20 to about 80 mg of fitusiran (e.g., about 20 mg, about 25 mg, about 30 mg, about 40 mg, about 50 mg, or about 80 mg). In some embodiments, the device may be used to deliver single dose of fitusiran, wherein the single dose comprises about 1 to about 30 mg of fitusiran (e.g., about 1.25 mg, about 2.5 mg, about 5 mg, about 10 mg, about 20 mg, or about 30 mg).

In one embodiment, the device may be used to deliver a single dose of about 80 mg of fitusiran. In one embodiment, the device may be used to deliver a single dose of about 50 mg of fitusiran. In one embodiment, the device may be used to deliver a single dose of about 20 mg of fitusiran. In one embodiment, the device may be used to deliver a single dose of about 30 mg of fitusiran. In one embodiment, the device may be used to deliver a single dose of about 10 mg of fitusiran. In one embodiment, the device may be used to deliver a single dose of about 5 mg of fitusiran. In one embodiment, the device may be used to deliver a single dose of about 2.5 mg of fitusiran. In one embodiment, the device may be used to deliver a single dose of about 1.25 mg of fitusiran.

In some embodiments, the single dose of fitusiran may be delivered in about 0.5 mL to about 1 mL delivery volumes (e.g., about 0.5 mL, about 0.6 mL, about 0.7 mL, about 0.8 mL, about 0.9 mL, or about 1 mL). Other delivery volumes described herein may also be used.

In one embodiment, the device may be used to deliver a single dose of about 80 mg of fitusiran in about 0.8 mL (about 100 mg fitusiran/mL). In one embodiment, the device may be used to deliver a single dose of about 50 mg of fitusiran in about 0.5 mL (about 100 mg fitusiran/mL). In one embodiment, the device may be used to deliver a single dose of about 20 mg of fitusiran in about 0.5 mL (about 40 mg fitusiran/mL). In one embodiment, the device may be used to deliver a single dose of about 30 mg of fitusiran in about 0.5 mL (about 60 mg fitusiran/mL). In one embodiment, the device may be used to deliver a single dose of about 10 mg of fitusiran in about 0.5 mL (about 20 mg fitusiran/mL). In one embodiment, the device may be used to deliver a single dose of about 5 mg of fitusiran in about 0.5 mL (about 10 mg fitusiran/mL). In one embodiment, the device may be used to deliver a single dose of about 2.5 mg of fitusiran in about 0.5 mL (about 5 mg fitusiran/mL). In one embodiment, the device may be used to deliver a single dose of about 1.25 mg of fitusiran in about 0.5 mL (about 2.5 mg fitusiran/mL).

In one embodiment, the device delivers fitusiran at a prophylactically effective amount to prophylactically treat hemophilia (e.g., hemophilia A or B, in a patient with or without inhibitors) in a patient in need thereof (e.g., a hemophilia A or B patient, with or without inhibitors). “Prophylactically effective amount” refers to the amount of fitusiran that helps the patient with hemophilia A or B, with or without inhibitors to achieve a desired clinical endpoint such as reducing the Annualized Bleeding Rate (ABR), Annualized Joint Bleeding Rate (AjBR), Annualized Spontaneous Bleeding Rate (AsBR), or the frequency of bleeding episodes. As used herein in the context of fitusiran, the term “treat” “treating,” or “treatment” includes prophylactic treatment of the disease and refers to achievement of a desired clinical endpoint.

A hemophilia A or B patient with inhibitors refers to a patient who has developed alloantibodies to the factor he/she has previously received (e.g., factor VIII for hemophilia A patients or factor IX for hemophilia B patients). A hemophilia A or B patient with inhibitors may become refractory to replacement coagulation factor therapies. A patient without inhibitors refers to a patient who does not have such alloantibodies. The present treatment methods may be beneficial for hemophilia A patients with inhibitors, as well as for hemophilia B patients with inhibitors.

As used herein, a patient with “hemophilia A or B, with or without inhibitors,” or refers to 1) a hemophilia A patient with inhibitors, or 2) a hemophilia B patient with inhibitors, 3) a hemophilia A patient without inhibitors, or 4) a hemophilia B patient without inhibitors. As used herein, a patient refers to a human patient. A patient can also refer to a human subject.

In some embodiments, the device may be used to prophylactically treat a patient with hemophilia A or B, with or without inhibitors, with a subcutaneous dose of about 50 mg of fitusiran once every two months (or every eight weeks). In other embodiments, the device may be used to prophylactically treat a patient with hemophilia A or B, with or without inhibitors, with a subcutaneous dose of about 50 mg of fitusiran every month (or every four weeks). In yet other embodiments, the device may be used to prophylactically treat a patient with hemophilia A or B, with or without inhibitors, with a subcutaneous dose of about 80 mg of fitusiran every two months (or every eight weeks). In yet other embodiments, the device may be used to prophylactically treat a patient with hemophilia A or B, with or without inhibitors, with a subcutaneous dose of about 80 mg of fitusiran every month (or every four weeks). In yet other embodiments, the device may be used to prophylactically treat a patient with hemophilia A or B, with or without inhibitors, with a subcutaneous dose of about 20 mg of fitusiran every two months (or every eight weeks). In yet other embodiments, the device may be used to prophylactically treat a patient with hemophilia A or B, with or without inhibitors, with a subcutaneous dose of about 20 mg of fitusiran every month (or every four weeks). In yet other embodiments, the device may be used to prophylactically treat a patient with hemophilia A or B, with or without inhibitors, with a subcutaneous dose of about 10 mg of fitusiran every month (or every four weeks). In yet other embodiments, the device may be used to prophylactically treat a patient with hemophilia A or B, with or without inhibitors, with a subcutaneous dose of fitusiran at about 30 mg every month (or every four weeks). In yet other embodiments, the device may be used to prophylactically treat a patient with hemophilia A or B, with or without inhibitors, with a subcutaneous dose of fitusiran at about 5 mg every month (or every four weeks). In yet other embodiments, the device may be used to prophylactically treat a patient with hemophilia A or B, with or without inhibitors, with a subcutaneous dose of fitusiran at about 2.5 mg every month (or every four weeks). In yet other embodiments, the device may be used to prophylactically treat a patient with hemophilia A or B, with or without inhibitors, with a subcutaneous dose of fitusiran at about 1.25 mg every month (or every four weeks).

Accordingly, provided herein is a method of prophylactic treatment of a patient with hemophilia A or hemophilia B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). Fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of prophylactic treatment of a patient with hemophilia A or hemophilia B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 mL PBS (at a concentration of about 100 mg fitusiran/mL).

Further provided herein is a method of reducing the frequency of bleeding episodes in a patient with hemophilia A or B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). Fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of reducing the frequency of bleeding episodes in a patient with hemophilia A or B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 mL PBS (at a concentration of about 100 mg fitusiran/mL).

Also, provided herein is a method of reducing the ABR in a patient with hemophilia A or B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). Fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of reducing the ABR in a patient with hemophilia A or B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 mL PBS (at a concentration of about 100 mg fitusiran/mL).

Also, provided herein is a method of reducing the AjBR in a patient with hemophilia A or B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). The fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of reducing the AjBR in a patient with hemophilia A or B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 mL PBS (at a concentration of about 100 mg fitusiran/mL).

Also, provided herein is a method of reducing the AsBR in a patient with hemophilia A or B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). Fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of reducing the AsBR in a patient with hemophilia A or B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 mL PBS (at a concentration of about 100 mg fitusiran/mL).

Any invention described herein is not limited by the description in conjunction with the exemplary embodiments. Rather, the invention and the associated disclosure comprise any new feature as well as any combination of features, particularly including any combination of features in the patent claims, even if said feature or said combination per se is not explicitly stated in the patent claims or exemplary embodiments. Further, as an alternative, the above drug delivery device 20 may comprise a pressure member, which works with air pressure, wherein the air pressure is used to squeeze the drug out of the pouch 24. For example, the pressure member may be or may comprise a pressure chamber, wherein an over- or under-pressure within the pressure chamber may be generated by the energy storage member 34 and wherein the pouch 24 is coupled to the pressure chamber such that the pressure within the pressure chamber may be used for squeezing the pouch 24. For example, the pressure chamber has an opening, which is sealingly covered by a wall of the pouch 24.

### REFERENCE NUMERALS

20 drug delivery device
22 housing
24 pouch
26 chamber
28 dispensing side
30 remote side
32 pressure member
34 energy storage member
35 drive portion
36 release mechanism
37 inner shoulder
38 drive portion recess
39 outer shoulder
40 dispensing direction
41 pin
44 chamfer
46 needle sleeve
48 needle sleeve spring
50 multi needle shield
52 needle arrangement
54 needles
56 pouch sealing member
58 gripping ring
60 needle sealing member
90 guide pin
92 torsion protection
94 flexible bars
96 first channel
98 bend
100 dead end
102 barb
104 second channel

1-20. (canceled)

21. A drug delivery device, comprising:
a housing for receiving a pouch within the housing, the pouch having collapsible walls enclosing a chamber, a dispensing side, and a remote side facing away from the dispensing side, and accommodating a drug within the chamber;
a needle arrangement comprising at least one needle configured to pierce the skin of a user, wherein the needle arrangement is configured to be brought into fluid communication with the dispensing side of the pouch;
a pressure member; and
an energy storage member coupled to the pressure member, the energy storage member being loaded in an initial state of the drug delivery device and being configured for squeezing the drug out of the chamber of the pouch by the pressure member during a dispensing operation of the drug delivery device.

22. The drug delivery device of claim 21, wherein the height of the pouch, that is the distance from the dispensing side to the remote side of the pouch, is smaller than the length of the pouch, that is the distance from one end of the dispensing side or the remote side to another end of the dispensing side or, respectively, the remote side.

23. The drug delivery device of claim 21, wherein the needle arrangement is configured to pierce the dispensing side of the pouch at a remote side of the needle arrangement and the skin at a dispensing side of the needle arrangement such that the needle arrangement communicates with the chamber and guides the drug under the skin during the dispensing operation.

24. The drug delivery device of claim 23, wherein the needle arrangement comprises at least two needles.

25. The drug delivery device of claim 24, wherein the needles are formed and arranged such that at least one of the needles provides a first injection depth and that at least one other of the needles provides a second injection depth.

26. The drug delivery device of claim 25, wherein the needle arrangement comprises at least three needles and wherein the needles providing the different injections depths are alternately arranged.

27. The drug delivery device of claim 24, wherein at least two of the needles have different lengths.

28. The drug delivery device of claim 24, wherein the needles are arranged as a linear array or as a planar array.

29. The drug delivery device of claim 21, further comprising a needle sleeve for protecting the needle arrangement in the initial state, the needle sleeve being operatively coupled to the energy storage member and being configured such that a release of the energy storage member is prevented as long as the needle sleeve protects the needle arrangement and that the energy storage member is released, when the needle sleeve exposes the needle arrangement.

30. The drug delivery device of claim 21, further comprising a flexible pouch sealing member which separates the needles from the pouch in the initial state and which is configured for being pierced by the needles for initiating the dispensing operation.

31. The drug delivery device of claim 21, further comprising a flexible needle sealing member which covers tips of the needles at their dispensing ends in the initial state and which is configured for being pierced by the needles for the dispensing operation.

32. The drug delivery device of claim 21, further comprising
a drive portion having a drive portion recess for accommodating the pressure member and the energy storage member, the drive portion being arranged on the remote side of the pouch; and
a release mechanism being configured for locking the pressure member and/or the energy storage member within the drive portion in the initial state and for releasing the pressure member and/or, respectively, the energy storage member for the dispensing operation.

33. The drug delivery device of claim 32, wherein the release mechanism comprises
a first release member at the drive portion, the first release member being configured to retain the pressure member in the initial state and to release the pressure member upon being activated; and
a second release member at the needle sleeve, the second release member being configured for activating the first release member for the dispensing operation.

34. The drug delivery device of claim 33, wherein the first release member comprises an inner shoulder of the drive portion, the inner shoulder extending from an inner wall of the drive portion recess radially inwardly and being configured to retain the pressure member in the initial state.

35. The drug delivery device of claim 34, wherein the first release member comprises an outer shoulder of the drive portion, the outer shoulder extending from an outer wall of the drive portion recess radially outwardly and being configured for being pushed radially outwardly by the second release member thereby pulling the inner shoulder radially outwardly to release the pressure member for initiating the dispensing operation.

36. The drug delivery device of claim 33, wherein
at least one of the first and second release member comprises a chamfer;
the other one of the first and second release member comprises a pin; and
the chamfer and the pin are formed and arranged such that the first release member is pressed outwardly by the second release member when the needle sleeve is moved into the housing.

37. The drug delivery device of claim 33, wherein the drive portion comprises the first release member, and/or wherein the needle sleeve comprises the second release member.

38. A drug delivery device, comprising:
a housing for receiving a pouch within the housing, the pouch having collapsible walls enclosing a chamber, a dispensing side, and a remote side facing away from the dispensing side, and accommodating a drug within the chamber;
a needle arrangement comprising at least one needle being configured to pierce the skin of a user, wherein the needle arrangement is configured to be brought into fluid communication with the dispensing side of the pouch;
a pressure member; and
an energy storage member coupled to the pressure member, the energy storage member being loaded in an initial state of the drug delivery device and being configured for squeezing the drug out of the chamber of the pouch by the pressure member during a dispensing operation of the drug delivery device, wherein the needle arrangement is configured to pierce the dispensing side of the pouch at a remote side of the needle arrangement and the skin at a dispensing side of the needle arrangement such that the needle arrangement communicates with the chamber and guides the drug under the skin during the dispensing operation, wherein the needle arrangement comprises at least two needles, and wherein the needles are arranged as a linear array or as a planar array.

39. The drug delivery device of claim 21, further comprising the pouch, wherein the pouch accommodates a drug within the chamber.

40. A method for dispensing a drug, the method comprising:
providing a drug delivery device, comprising
a pouch having collapsible walls enclosing a chamber, a dispensing side, and a remote side facing away from the dispensing side, and accommodating the drug within the chamber;
a housing for receiving the pouch within the housing;
a needle arrangement comprising at least one needle configured to pierce the skin of a user, wherein the needle arrangement is configured to be brought into fluid communication with the dispensing side of the pouch;
a pressure member; and
an energy storage member coupled to the pressure member, the energy storage member being loaded in an initial state of the drug delivery device and being configured for squeezing the drug out of the chamber of the pouch by the pressure member during a dispensing operation of the drug delivery device,
a drive portion having a drive portion recess for accommodating the pressure member and the energy storage member, the drive portion being arranged on the remote side of the pouch; and
a release mechanism being configured for locking the pressure member and/or the energy storage member within the drive portion in the initial state and for releasing the pressure member and/or, respectively, the energy storage member for the dispensing operation, and
activating the release mechanism to release the pressure member.
